(19) United States
(12) Patent Application Publication
(10) Pub. No.: US 2022/0287566 A1
(43) Pub. Date: Sep. 15, 2022

(54) SYSTEM FOR PROVIDING TARGETED ELECTRICAL STIMULATION TO TISSUE

(71) Applicant: CHECKPOINT SURGICAL, INC., Cleveland, OH (US)

(72) Inventors: Robert B. Strother, Willoughby Hills, OH (US); Jonathan L. Sakai, Fairview Park, OH (US)

(21) Appl. No.: 17/831,467

(22) Filed: Jun. 3, 2022

Related U.S. Application Data
(63) Continuation of application No. 13/606,862, filed on Sep. 7, 2012, now abandoned.
(60) Provisional application No. 61/532,550, filed on Sep. 8, 2011.

(51) Int. Cl. A61B 5/00; A61N 1/05; A61N 1/36

(52) U.S. Cl. CPC A61B 5/0048; A61N 1/0551; A61N 1/36017

(57) ABSTRACT

Provided is a system that may be used in delivering targeted electrical stimulation to tissue, such as animal bodily tissue. A system according to the present invention includes a longitudinal stimulation probe that includes a first electrode disposed at a distal tip and a second electrically conductive surface spaced proximally from the first electrode. Another system according to the present invention may include an improved user interface including an improved probe/handle interface including high visibility indication.

### RELATED APPLICATIONS

This application claims the benefit of U.S. Provisional Patent Application Ser. No. 61/532,550, filed 8 Sep. 2011, and entitled “System for Providing Targeted Electrical Stimulation to Tissue,” which is incorporated by reference herein in its entirety.

### BACKGROUND OF THE INVENTION

Embodiments according to the present invention relate to electrical stimulation devices, and more particularly to configurations of electrical stimulation probes and communication of information from an electrical stimulation device.

Prior electrical stimulation apparatuses are known. Generally, however, only a portion of the probe's conductive surface (the end of the tip in contact with the tissue and the small meniscus of body fluid that forms to that tip) delivers stimulus current to tissue as the device is used in open surgical procedures. In arthroscopic procedures, however, at least some of the probe, and perhaps the entire probe, may be exposed to fluid (nominally saline), leading to concerns consistent with informal intraoperative experience that the stimulus current may be shunted away from the 5 mm long conductive tip by the surrounding saline. Such shunting reduces the current flowing from the tip into the tissue to a level too low to desirably activate target nerves innervating the tissue, even when the probe is contacting tissue directly over the target nerve.

### SUMMARY OF THE INVENTION

Embodiments of a probe according to the present invention include a cathode located on a distal tip of a probe and a proximally located anode on the probe, which is spaced some insulative distance from the cathode. A preferred cathode has a conductive surface area of preferably less than or equal to ten square millimeters. A preferred anode provides a cylindrical electrically conductive surface that is greater in area than the exposed, electrically conductive surface of the cathode, preferably greater than nine times that of the cathode. The insulative distance between the exposed electrically conductive cathode surface and the exposed electrically conductive anode surface is preferably at least three millimeters and more preferably five to ten millimeters.

Probe constructions according to the present invention may be beneficially exploited in arthroscopic procedures, in which targeted electrical stimulation is desirable to be applied to tissue that may be covered in a pool or volume of conductive solution, such as saline solution. Preferably, in use, both electrode surfaces would be surrounded by saline which was previously or simultaneously infused to fill a surgical site. Alternatively, only a portion of the electrically conductive anode surface may be disposed within the saline solution. Preferably, all probe features are sized to enable passage through a 5 mm (inner diameter) arthroscopic surgical cannula. Although both electrodes are physically near one another, the construction and usage are such that the probe generates an essentially monopolar stimulating field. Key construction & usage details include: the Anode (return) sleeve is spaced at least 3 mm away from the Cathode at the end of the probe, the sleeve is surrounded by saline, the sleeve has a much greater surface area than the Cathode, and the sleeve is usually not directly touching excitable tissue.

A system according to the present invention may include an improved user interface disposed at or near a junction point of a probe section of an electrical stimulator and a handle section thereof.

### BRIEF DESCRIPTION OF THE DRAWINGS

FIG. 1A is a perspective view of an electrical stimulator incorporating embodiments according to the present invention.

FIG. 1B is a perspective close-up view of a probe construction according to the present invention.

FIG. 2A is a cross-section view of a first embodiment of a probe construction according to the present invention taken along line 2A-2A of FIG. 1B.

FIG. 2B is a cross-section view of a second embodiment of a probe construction according to the present invention.

FIG. 2C is a cross-section view of a third embodiment of a probe construction according to the present invention.

FIG. 3 is a partial cutaway right elevation view of the embodiment of FIG. 1A.

FIG. 4 is a partial cutaway cross-section view taken along line 4-4 of FIG. 1A.

FIG. 5 is a partial cutaway cross-section view taken along line 5-5 of FIG. 4.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention.

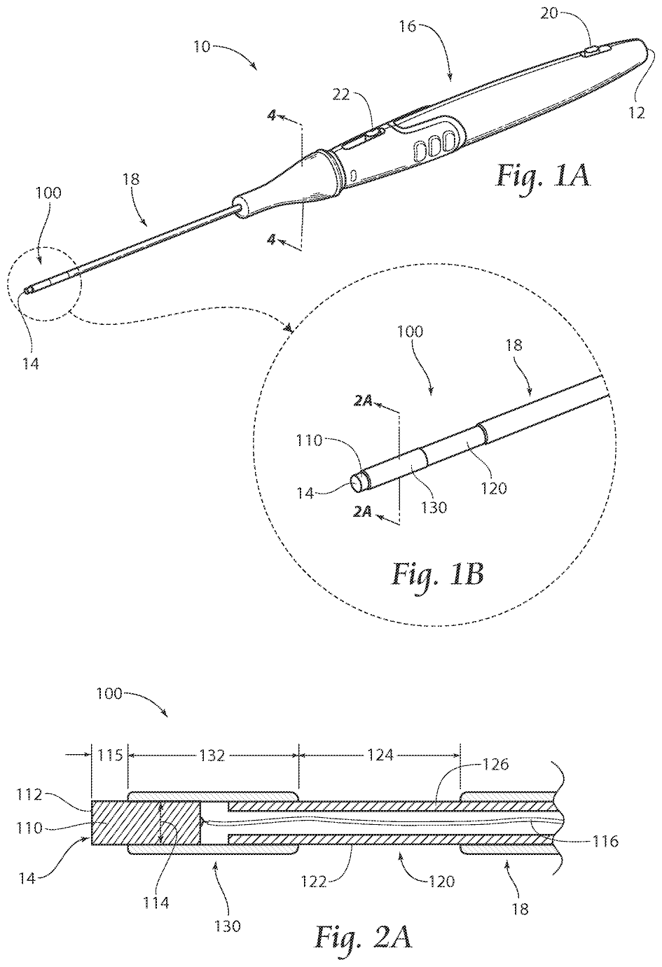

Turning now to the figures, FIG. 1A depicts an electrical stimulator device 10 incorporating embodiments according to the present invention. The stimulator 10 extends from a proximal end 12 to a distal end 14. A handle 16 extends from the proximal end 12 towards the distal end 14. The handle 16 preferably houses electrical stimulation generation circuitry, which may receive control inputs from user input controls, such as an amplitude adjustment switch 20 and/or a preferably continuous pulse width adjustment slide switch 22. The handle 16 may also include a low (relative to the rest of the handle 16) durometer grip portion 24 to aid in manual manipulation.

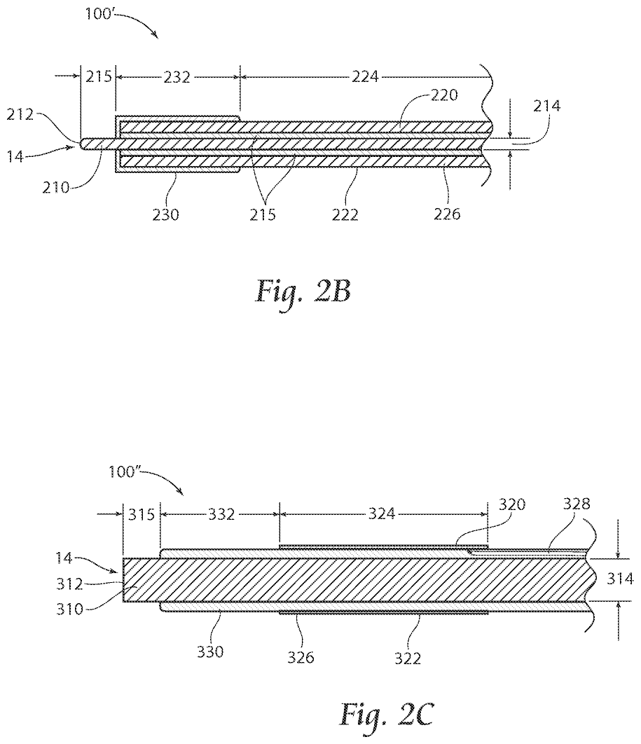

A probe 18 extends from the distal end 14 towards the proximal end 12 and is coupled to the handle 16. Provided at and extending from the distal end 14 towards the proximal end 12 is a preferred probe configuration 100, as seen in FIG. 1B. With reference also to FIG. 2A, a preferred probe configuration 100 according to the present invention includes a first electrode 110, a second electrode 120, and a first insulative portion 130. The first electrode 110 has a first external electrically conductive surface 112, having a first exposed electrode surface area, which is disposed at the distal end 14 of the probe 18. The electrically conductive surface 112 is electrically coupled to the electrical stimulation generation circuitry that is housed in the handle 16. The first surface 112, which preferably serves as a stimulation cathode, has a preferred diameter 114, such as about two to about three millimeters, and more preferably about 2.5 mm. The second electrode 120 has a second external electrically conductive surface 122 having a second exposed electrode surface area. The second conductive surface 122, which is preferably at least substantially cylindrical, is spaced proximally from the first conductive surface 112 by the first insulative portion 130. The second conductive surface 122 may extend from the first insulative portion 130 for an electrode length 124. The first conductive surface 112 and the second conductive surface 122 may be formed of any desirable material, but preferably are stainless steel.

The first conductive surface 112 also preferably extends along a desirable length 115, longitudinally distally from the first insulative portion 130. A preferred range of lengths 115 is from 0.00 millimeters to about 6.00 millimeters, with about 0.50 to about 5.10 millimeters being most preferred. The length 115 may be selected on the basis of a desired expected charge density to be experienced during use. Preferred cathodic charge density may be in the range of about 0.5 μC/mm2 to about 2.0 μC/mm2, while preferred anodic charge density may be in the range of greater than zero μC/mm2 to about 0.6 μC/mm2. During use, it may be desirable to prevent tissue damage, in which case the size of the first electrode 110 and/or second electrode 120 should be sized accordingly. A preferred surface area of a cathodic surface, either the conductive surface 112 of the first electrode 110 or the conductive surface 122 of the second electrode 120, is generally about 1 mm² to about 20 mm², with about 1.6 mm² to about 16 mm² being most preferable.

Preferably, the second exposed surface area of the second conductive surface 122 is greater than the first exposed surface area of the first conductive surface 112. More preferably, for an expected depth of saline solution disposed on a stimulation side of the animal tissue to be stimulated, a greater amount of the second surface area is exposed to the solution than the whole of the first surface area. For example, if the first exposed surface area of the first conductive surface 112 is about seven square millimeters, then the electrode length 124 should be selected so as to establish a second exposed surface area of the second conductive surface 122 that is greater than seven square millimeters. Even more preferably, the second exposed surface area of the second conductive surface 122 is more than five times the first exposed surface area, and most preferably, it is at least nine times the first exposed surface area. Thus, in our example, if the first surface area is seven square millimeters, the second surface area is most preferably at least 63 square millimeters.

The second conductive surface 122 may be formed as an external surface of a conductive cylinder or other tubular member 126, which may extend a majority of a longitudinal length of the probe 18. The second conductive surface 122 is electrically coupled to the electrical stimulation generation circuitry contained within the handle 16, and the surface 122 preferably serves as a stimulation anode. If the member 126 is used, the first conductive surface 112 may be coupled to the stimulation circuitry by an insulated, electrically conductive filament or wire 116, which is soldered or otherwise electrically coupled to the first electrode 110.

The first insulative portion 130 is disposed proximal to the first conductive surface 112 and operates to prevent direct proximal conduction of electrical current between the first conductive surface 112 and the second conductive surface 122 if the probe 18 is disposed in air. The first insulative portion 130 provides a minimum insulative gap 132 between the first conductive surface 112 and the second conductive surface 122. The minimum insulative gap 132 is preferably three millimeters, but even more preferably five millimeters.

A second preferred probe configuration 100′ is shown in FIG. 2B, where like numbers refer to like structure from the first embodiment 100. This embodiment 100′, like the first embodiment 100, has a first electrode 210, a second electrode 220, and a first insulative portion 230. The dimensions of the second embodiment 100′ are preferably similar to or the same as the first embodiment 100, but the diameter 214 of the first conductive surface 212 may be less than 2.5 mm, such as about 1 mm.

The first conductive surface 212 also preferably extends along a desirable length 215, longitudinally distally from the first insulative portion 230. A preferred range of lengths 215 is from 0.00 millimeters to about 6.00 millimeters, with about 0.50 to about 5.10 millimeters being most preferred. The length 215 may be selected on the basis of a desired expected charge density to be experienced during use. Preferred cathodic charge density may be in the range of about 0.5 μC/mm2 to about 2.0 μC/mm2, while preferred anodic charge density may be in the range of greater than zero μC/mm2 to about 0.6 μC/mm2. During use, it may be desirable to prevent tissue damage, in which case the size of the first electrode 210 and/or second electrode 220 should be sized accordingly. A preferred surface area of a cathodic surface, either the conductive surface 212 of the first electrode 210 or the conductive surface 222 of the second electrode 220, is generally about 1 mm² to about 20 mm², with about 1.6 mm² to about 16 mm² being most preferable.

The method of construction of the second embodiment 100′ differs slightly from the first embodiment 100, however. While the second electrode 220 may be formed generally as a conductive tubular member 226, the first electrode 210 may be extend throughout at least a majority of the tubular member 226, but electrically insulated from contact therewith by an insulative sheath or layer 215. At the distal end of the tubular member 226, the insulative portion 230 preferably extends over such distal end and along the minimum insulative gap distance 232.

A third preferred probe configuration 100″ is shown in FIG. 2C, where like numbers refer to like structure from the first embodiment 100. This embodiment 100″, like the first embodiment 100, has a first electrode 310, a second electrode 320, and a first insulative portion 330. The dimensions of the second embodiment 100″ are preferably similar to or the same as the first embodiment 100. The method of construction of the third embodiment 100″ differs slightly from the first embodiment 100, however. The third embodiment 100″ includes a partially insulated conductor with an exposed tip that forms the first electrode 310 and the first insulative portion 330. The second electrode 320 is provided as a conductive sleeve 326, of a desired length 324, such as about 10 millimeters (mm), that may be crimped or otherwise fastened to the insulator of the probe 18. The second electrode 320 is electrically coupled to the electrical stimulation generation circuitry through an electrical filament or wire 328, which may be disposed within or coupled to the insulated portion of the sheath 18.

The first conductive surface 312 also preferably extends along a desirable length 315, longitudinally distally from the first insulative portion 330. A preferred range of lengths 215 is from 0.00 millimeters to about 6.00 millimeters, with about 0.50 to about 5.10 millimeters being most preferred. The length 315 may be selected on the basis of a desired expected charge density to be experienced during use. Preferred cathodic charge density may be in the range of about 0.5 μC/mm2 to about 2.0 μC/mm2, while preferred anodic charge density may be in the range of greater than zero μC/mm2 to about 0.6 μC/mm2. During use, it may be desirable to prevent tissue damage, in which case the size of the first electrode 310 and/or second electrode 320 should be sized accordingly. A preferred surface area of a cathodic surface, either the conductive surface 312 of the first electrode 310 or the conductive surface 322 of the second electrode 320, is generally about 1 mm² to about 20 mm², with about 1.6 mm² to about 16 mm² being most preferable.

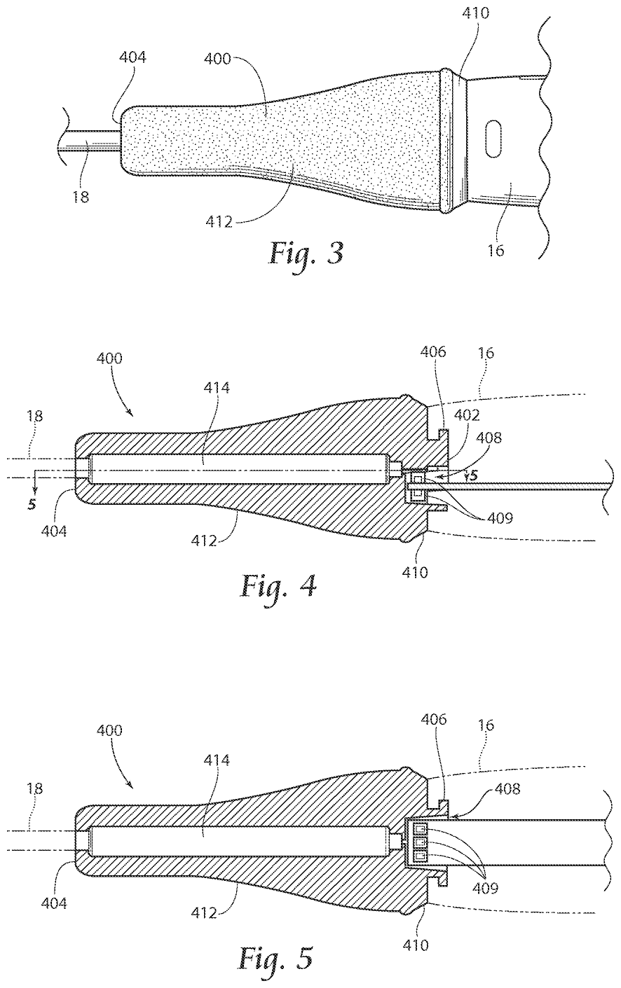

Turning now to FIGS. 3-5, an improved user interface may be described. The improved user interface comprises an improved status indicator interface including a shroud 400, which may be provided at and preferably surrounding or spanning the mechanical coupling of the probe 18 to the handle 16. The shroud 400 extends from a proximal mounting end 402 to a distal probe end 404. At the mounting end 402, the shroud 400 preferably includes a mounting flange or clip 406 to interface with and be captured by a portion of the handle 16. Alternatively, the shroud 400 may be formed integrally with the handle 16. Preferably formed longitudinally into the mounting end 402 is an indicator port 408 which may be adapted to at least partially surround one or more light sources 409, such as one or more light emitting diodes that are electrically coupled to the electrical stimulation generation circuitry in the handle 16. Disposed radially outwardly from the indicator port 408, and preferably on the external surface of the shroud 400, is a frustoconical lens surface 410, which may be polished. The frustoconical surface 410 helps to direct light from one or more light source 409 disposed at least partially within the indicator port 408 proximally towards the proximal end 12 of the device 10. The surface 410 preferably extends about an entire circumference of the shroud 400, though only a portion of the circumference may be used. The shroud 400 may also be provided without the frustoconical lens surface 410. The shroud 400 may be molded or otherwise formed, preferably as a unitary member, that is transparent or translucent. While the frustoconical surface 410 may be polished to enhance emission, the remainder of the external surface 412 of the shroud 400 is preferably textured in some way, such as by being bead-blasted, so as to assist in diffusion of light generated by the light sources 409. One or more of the indicated features assists in user visibility of light indications generated by the device. Formed into the shroud 400 is a probe bore 414 which allows passage of the probe 18 and/or electrical conductors (e.g. 116) into the shroud 400 to pass through to the circuitry in the handle 16.

The operation of one or more light sources 409 as a status indicator may be accomplished in a number of ways. For instance, visual indication provided by one or more light source 409 may allow an operator of the device 10 to confirm delivery or transmission of stimulus current. Through the use of different light colors, different flash rates, etc., the improved user interface may allow the operator to confirm that the instrument is powered on, and whether stimulus current is flowing. Thus the operator may have a greater confidence that, where there exists a failure to elicit a desired neurological response (e.g., a muscle contraction), such failure is more likely because of lack of viable nervous tissue near or contacted by the tip (e.g. 112) of the stimulator 50 rather than equipment malfunction, power failure, or other instrumentation problems.

As a representative example, one or more, or all light sources 409 may be configured to illuminate continuously in one color when the power is applied to the electrical stimulation circuitry within the handle 16 but the probe 18 is not in contact with tissue. After contact with tissue is made, one or more light sources 409 may flash (i.e., blink) or pulsate (i.e., change brightness intensity by dimming and/or brightening) to indicate that stimulation is available for delivery (i.e.

stimulation would flow between the first electrode 110 and second electrode 120 if sufficient conductivity therebetween is established). If the stimulation has been requested, (i.e., the stimulation is available for delivery, but there is no stimulation being delivered because of a lack of sufficient conductivity between the first electrode 110 and the second electrode 120), one or more light sources 409 may illuminate in a different color, and/or may illuminate continuously or may flash.

By way of further example, one or more light sources 409 may operate in the following manner. One or more light sources 409 may provide a first indication that the stimulator 10 has not yet been turned on, that the stimulator power source has been depleted, that the stimulator has been used beyond some predetermined life span, or that an error has occurred thereby preventing stimulation. One or more light sources 409 may provide a second indication that the stimulator has been activated or powered on, but further that stimulation is not available for delivery. One or more light sources 409 may provide a third indication that the stimulator has been activated or powered on, and that greater than or equal to a first predetermined threshold of stimulus current is verified as being conducted between the first electrode and the second electrode. One or more light sources 409 may provide a third indication that the stimulator has been activated or powered on, and that not more than a second predetermined threshold of stimulus current is verified as being conducted between the first electrode and the second electrode.

The mentioned indications provided by one or more light sources 409 may include display styles such as the light sources being off (i.e. not emitting light), a single color at a single intensity, intermittency (blinking) of a single color at a substantially single intensity, a single color at a varying intensity (pulsing), intermittency of a single color at varying intensity (pulsed blinking), or similar operation using a plurality of colors. For instance, the first indication is preferably provided by keeping all light sources off (i.e. not emitting any light). The second indication is preferably provided by one or more, or all, light sources 409 emitting a yellow light at a single intensity. The third indication is preferably provided by one or more, or all, light sources 409 emitting a blinking yellow light at a substantially single intensity. The third indication is preferably provided by one or more, or all, light sources 409 emitting a blinking red light at a substantially single intensity.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention.

1-15. (canceled)

16. An electrical stimulator comprising:
a body;
an electrical stimulation circuitry at least partially disposed within the body, the electrical stimulation circuity configured to provide an electrical stimulation;
a probe coupled to and extending away from the body;
a first electrode disposed at a distal end of the probe, wherein the first electrode is in electrical communication with the electrical stimulation circuitry; and
a second electrode disposed on the probe at a location proximal to the first electrode, wherein the second electrode is electrically insulated from the first electrode by an insulated portion positioned between the first and second electrodes and wherein the second electrode is in electrical communication with the electrical stimulation circuitry, wherein the first and second electrodes generate a monopolar stimulation field and are sized to prevent tissue damage during the electrical stimulation.

17. The electrical stimulator of claim 16 further comprising a visual status indicator comprising a shroud mounted to and extending from the handle and at least one light source disposed within the shroud, wherein the light source confirms delivery of the electrical stimulation.

18. The electrical stimulator of claim 17, wherein at least a portion of the probe passes through an aperture of the shroud.

19. The electrical stimulator of claim 17, wherein the shroud extends away from the body and towards the probe, and wherein the shroud comprises an exterior surface that is at least partially translucent.

20. The electrical stimulator of claim 16, wherein the first and second electrodes are cylindrical in shape.

21. The electrical stimulator of claim 16, wherein the first electrode is a solid cylinder.

22. The electrical stimulator of claim 16, wherein the second electrode is cylindrical in shape and comprises an aperture formed therethrough, and wherein the second electrode is displaced from the distal end of the probe.

23. The electrical stimulator of claim 16, wherein the probe is straight along at least its entire length and the first and second electrodes are disposed coaxially with each other.

24. The electrical stimulator of claim 23, wherein the first electrode has a first exposed surface area and the second electrode has a second exposed surface area, and further wherein the second exposed surface area is greater than the first exposed surface area.

25. The electrical stimulator of claim 24, wherein the second exposed surface area is five times to nine times the first exposed surface area.

26. The electrical stimulator of claim 16, wherein the first electrode comprises a length longitudinally distally from a first insulative portion on said probe, wherein said length relates to an expected charge density during application of the electrical stimulation.

27. An electrical stimulation device comprising:
a body comprising an electrical stimulation circuitry; and
a longitudinal probe extending from the body and supporting first and second electrodes, the first electrode being disposed at and including a distal tip of the probe, and the second electrode being positioned proximal to the first electrode and electrically insulated therefrom by an insulated portion positioned between the first and second electrodes;
wherein the first electrode is configured for delivering electrical stimulation to a target bodily tissue in an animal body; and
the second electrode is configured for receiving at least a portion of the electrical stimulation that has been conducted at least partially through the target bodily tissue, wherein the first and second electrodes are sized to prevent tissue damage during the electrical stimulation and wherein the first and second electrodes generate a monopolar stimulation field during delivery of the electrical stimulation.

28. The electrical stimulation device of claim 27 further comprising a visual status indicator that extends from the handle, the visual status indicator comprising:
a shroud; and
a light source disposed within the shroud and in electrical communication with the electrical stimulation circuitry;

29. The electrical stimulation device of claim 28, wherein the first electrode comprises a length longitudinally distally from said insulated portion, wherein said length relates to an expected charge density during application of the electrical stimulation.

30. The electrical stimulation device of claim 29, wherein the electrical stimulation circuitry is configured for generating an electrical charge at the first electrode at a charge density level of 0.5 to 2.0 micro-coulombs.

31. The electrical stimulation device of claim 28, wherein the light source is configured to generate an indication in response to verifying that a threshold amount of stimulus current associated with the electrical stimulation has been received by the second electrode.

32. The electrical stimulation device of claim 28, wherein the light source is configured to generate an indication in response to at least one of the first electrode not contacting the target bodily tissue or the second electrode not contacting the target bodily tissue.

33. An electrical stimulation device comprising:
a body comprising electrical circuitry;
a visual status indicator extending from the body, wherein the visual status indicator comprises;
a shroud; and
a light source disposed within the shroud and in electrical communication with the electrical stimulation circuitry;
a probe extending from and passing at least partially through the shroud;
a first electrode disposed at a distal end of the probe;
an insulative portion extending longitudinally from the first electrode; and
a second electrode electrically insulated from the first electrode by the insulative portion and disposed on the probe at a location longitudinally proximal to the electrode,
wherein the first electrode and the second electrode are in electrical communication with the electrical circuitry and the first electrode and the second electrodes are sized to prevent tissue damage during electrical stimulation and the first and second electrodes generate a monopolar stimulation field during delivery of electrical stimulation.

34. The electrical stimulation device of claim 33, wherein the at least one light source generates an indication in response to at least one of an amount of stimulus current received by the second electrode, a status of a power source, a predetermined life span, or an output of the electrical circuitry.

35. The electrical stimulation device of claim 33, wherein the second electrode comprises an exposed surface area at least one to five times larger than an exposed surface area of the first electrode.

36. The electrical stimulation device of claim 33, wherein the probe is sized to pass through an arthroscopic cannula having an inner diameter of 5 mm.
